SEQUENCE LISTING (2 sequences)

SEQ ID NO: 1 - protein, 26 aa, Homo Sapiens
VETRTDVVFDLPFLMIIDPTDSQDIF

SEQ ID NO: 2 - protein, 25 aa, Homo Sapiens
  misc._feature (4)..(4): Xaa represents unknown amino acid, most likely Leu
LVKXGIKRFAGPFSIIVPTDSEDIF

(19) United States
(12) Patent Application Publication
(10) Pub. No.: US 2011/0097747 A1
(43) Pub. Date: Apr. 28, 2011

(54) Monoclonal antibody binds antigen associated with human tumors

(76) Inventor: Kevin Jones, Knoxville, TN (US)

(21) Appl. No.: 12/589,588

(22) Filed: Oct. 26, 2009

(51) Int. Cl. G01N 33/53; C07K 16/00; C12N 9/96; C12N 5/00; C07K 14/00

(52) U.S. Cl. 435/7.92; 530/387.9; 530/391.3; 435/188; 530/387.3; 436/501; 435/7.9; 435/325; 530/350

(57) ABSTRACT

The present invention is concerned with a novel antigen associated with human tumors, including carcinomas of the colon, pancreas, and lung, as well as novel monoclonal antibodies which binds strongly to said antigen. The antibodies bind to normal human cells to a much lesser degree than to tumor cells. The antibodies find use both in diagnostic methods such as the detection of malignant cells associated with tumors and in therapeutic methods for treatment of humans with tumors. The novel antigen disclosed is a 60,000 Dalton glycoprotein found on the cell surface of human tumor cells.

### FIELD OF THE INVENTION

The present invention relates to two novel monoclonal antibodies reactive with human carcinoma cells and to methods for production and use of such novel monoclonal antibodies. More specifically, the monoclonal antibodies of this invention are reactive with a novel antigen which is associated with a variety of human tumors including carcinomas of the colon, lung, and pancreas.

The monoclonal antibodies of this invention are useful for diagnostic and therapeutic purposes. Uses include detecting elevated humoral levels of prognostic antigens, in vivo and in vitro imaging of malignant carcinomas, stimulation and programming of immune responses.

### SEQUENCE LISTING

A sequence listing is part of this application.

### BACKGROUND OF THE INVENTION

Carcinomas cause millions of deaths annually. Of all cancers, colorectal cancer is the second leading cause of cancer-related deaths in the U.S., while pancreatic cancer is the eleventh most common cancer and the fourth leading cause of cancer death in both men and women. Most cases of carcinomas are incurable by chemotherapy and radiation therapy unless detected and treated in the early stages of the disease. The more advanced a cancer is when it is diagnosed, the less likely it is that therapy will be effective. Therefore, despite the advances in cancer research, there remains a need for novel monoclonal antibodies useful for the early diagnosis and treatment of carcinomas of the colon, pancreas and lung.

Generally, monoclonal antibodies are used as invaluable reagents in diagnostics. In fact, they have played a major role in deciphering the functions of various bio-molecules in biosynthetic pathways. They have also become the reagents of choice for identification and characterization of tumor specific antigens and have become a valuable tool in the classification of cancer.

Once tumor-associated antigens have been purified from tissue extracts such antigens can be used to elicit production of antibodies to the antigen by injection into animals. Monoclonal antibodies can then be produced via hybridoma fusion techniques. (see, Kohler and Milstein, Nature, 256:495-97 (1975); Brown et al., J. Immunol., 127 (2):539-46 (1981); Brown et al., J. Biol. Chem., 255:4980-83 (1980); Yeh et al., Proc. Nat'l. Acad. Sci. (USA), 76 (6):2927-31 (1976); and Yeh et al., Int. J. Cancer, 29:269-75 (1982)).

Monoclonal antibodies reactive with carcinoma-associated antigens are known (see, e.g., Papsidero, Semin. Surg. Oncol., 1 (4):171-81 (1985); Schlom et al., Important Adv. Oncol., 170-92 (1985); Allum et al., Surg. Ann., 18:41-64 (1986); Houghton et al., Semin. Oncol., 13 (2):165-79 (1986); Monoclonal Antibodies in Cancer: Advances for Diagnosis and Treatment, Roth (ed.), Futura Publishing, Mt. Kisco, N.Y. (1986); and Cancer Diagnosis In Vitro Using Monoclonal Antibodies, Kupchik (ed.) Marcel Dekker, Inc., New York, (1988)).

Many of the known monoclonal antibodies are reactive with several types of human carcinomas, while a few antibodies react with carcinomas derived from specific organs of the body, e.g., lung, breast, ovary, colon, stomach or pancreas. (See, e.g., Fink et al., Prog. Clin. Pathol., 9:121-33 (1984)). For example, monoclonal antibodies reactive with glycoprotein antigens on specific types of carcinomas include those described in U.S. Pat. No. 4,737,579 (monoclonal antibodies to non-small cell lung carcinomas); U.S. Pat. No. 4,753,894 (monoclonal antibodies to human breast cancer); U.S. Pat. No. 4,579,827 (monoclonal antibodies to human gastrointestinal cancer); and U.S. Pat. No. 4,713,352 (monoclonal antibodies to human renal carcinoma).

Monoclonal antibodies reactive with glycolipid antigens that are believed to be associated with certain tumor cells have also been disclosed. For example, see Kniep et al., J. Immunol., 131 (3):1591-94 (1983) and U.S. Pat. No. 4,507,391 (monoclonal antibody to human melanoma).

In addition, monoclonal antibodies reactive with glycolipid antigens found on specific types of carcinoma cells include those described by Rosen et al., Cancer Research, 44:2052-61 (1984) (monoclonal antibodies to human small cell lung cancer); Varki et al., Cancer Research, 44:681-87 (1984) (monoclonal antibodies to human adenocarcinomas of the lung, stomach and colon and melanoma); and U.S. Pat. No. 4,579,827 (monoclonal antibodies to human colon adenocarcinoma).

Additional monoclonal antibodies exhibiting a reactivity to antigens associated with tumor cells are greatly needed.

In vitro diagnostic methods are known in the art and include immunohistological detection of tumor cells (e.g., on human tissue, cells or excised tumor specimens) or serologic detection of tumor-associated antigens (e.g., in blood samples or other biological fluids). Immunohistological techniques involve contacting a biological specimen such as a tumor tissue specimen with the antibody of the invention and then detecting the presence on the specimen of the antibody complexed to its antigen. The formation of such antibody-antigen complexes with the specimen indicates the presence of tumor cells in the tissue. Detection of the antibody on the specimen can be accomplished using techniques known in the art, such as the immunoperoxidase staining technique, the avidin-biotin (ABC) technique or immunofluorescence techniques (see Ciocca et al., Meth. Enzymol., 121:562-79 (1986); Kimball (ed.), Introduction To Immunology (2nd Ed.), pp. 113-117, Macmillan Publ. Co. (1986)).

Serologic diagnostic techniques involve the detection and quantitation of tumor-associated antigens that have been secreted or “shed” into the serum or other biological fluids of patients thought to be suffering from carcinoma. Such antigens can be detected in the body fluids using techniques known in the art such as radioimmunoassays (RIA) or enzyme-linked immunosorbent assays (ELISA) wherein an antibody reactive with the “shed” antigen is used to detect the presence of the antigen in a fluid sample (see, e.g., Uotila et al., J. Immunol. Methods, 42:11 (1981); Allum et al., “Monoclonal Antibodies in the Diagnosis and Treatment of Malignant Conditions” Surg. Ann., 18:41-64, 48-51 (1986); Sikora et al. (eds.), Monoclonal Antibodies, pp. 32-52, Blackwell Scientific Publications, (1984)).

It is thus apparent that a monoclonal antibody reactive with an antigen expressed at high levels by a variety of tumors may become useful towards an earlier diagnosis of cancers, the immunological monitoring of cancer patients, as well as for development of improved methods for therapy of cancers. It is also apparent that purified antigens associated with carcinomas derived from specific organs of the body can be of value for creating such monoclonal antibodies, as well as for creating cancer vaccines.

### SUMMARY OF THE INVENTION

An object of the present invention provides for monoclonal antibodies, or portions of monoclonal antibodies (peptides) having specificity directed to epitopes of human lung, colorectal, and pancreatic carcinoma-associated antigen. The present invention provides two such monoclonal antibodies, the CA11-19 antibodies, which specifically bind a particular antigen, the EDP antigen, which is associated with human tumor cells, particularly cells from lung, pancreas, and colon carcinomas. Thus, the antibodies of the invention can be useful for the diagnosis and therapy of tumors associated with the EDP antigen identified by the CA11-19 antibodies. The CA11-19 antibodies of the invention show no significant reactivity with normal human cells.

The antibodies of the present invention may be used for in-vitro diagnostic methods for determining the presence of a malignant condition in human colon tissue and other human tissues. One such method involves using the antibody, antibodies, or antibody fragments of the present invention in an ELISA assay. Another such method involves using the antibody, antibodies, or antibody fragments of the present invention for detection of tumor cells within biopsied tissue through the use of immunohistochemistry techniques.

Another object of the present invention provides in-vivo diagnostic methods for the localization of a tumor by administering to a patient a purified antibody, antibodies, or antibody fragments of the present invention labeled with an agent which gives a detectable signal. The localization is then detected using external scintigraphy, emission tomography, radionuclear scanning, or other known methods of imaging.

The invention also has therapeutic applications, since the CA11-19 antibodies and similar antibodies can react with the EDP antigen that is expressed in high concentrations at the tumor cell surface. The monoclonal antibody of the invention may be used in immunoconjugates as a carrier of various agents which have an antitumor effect, including, but not restricted to, chemotherapeutic drugs, toxins, immunological response modifiers, and radioisotopes.

Another object of the present invention provides methods for purifying EDP antigen.

### DETAILED DESCRIPTION OF INVENTION

In order that the invention herein described may be more fully understood, the following detailed description is set forth.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an, and she” include the plural reference unless the context clearly indicates otherwise. Thus, for example, the reference to an antibody is a reference to one or more such antibodies, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages may mean ±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains.

For the purposes of this application the following terms shall have the following meanings:

An “epitope” refers to that portion of any molecule capable of being recognized by, and bound by, an antibody (the corresponding antibody binding region may be referred to as a paratope). In general, epitopes consist of chemically active surface groupings of molecules, for example, amino acids or sugar side chains, and have specific three-dimensional structural characteristics as well as specific charge characteristics.

An “antigen” is a molecule or a portion of a molecule capable of being bound by an antibody. An antigen may have one or more than one epitope. An antigen will bind in a highly selective manner with its corresponding antibody and not with the multitude of other antibodies which may be evoked by other antigens.

An “antibody” includes both intact immunoglobulin molecules as well as portions, fragments, peptides and derivatives thereof, such as, for example, Fab, Fab′, F(ab′)2, Fv, CDR regions, or any portion or peptide sequence of the antibody that is capable of binding antigen or epitope. An antibody is said to be “capable of binding” a molecule if it is capable of specifically reacting with the molecule to thereby bind the molecule to the antibody.

Antibody also includes chimeric antibodies, anti-idiotypic (anti-Id) antibodies to antibodies that can be labeled in soluble or bound form, as well as fragments, portions, regions, peptides or derivatives thereof provided by any known technique, such as, but not limited to, enzymatic cleavage, peptide synthesis, or recombinant techniques. Such antibodies of the present invention are capable of binding portions of EDP antigen or EDP antigen-bearing cells. Antibody fragments or portions may lack the Fc fragment of intact antibody, clear more rapidly from the circulation, and may have less non-specific tissue binding than an intact antibody. Examples of antibody may be produced from intact antibodies using methods well known in the art, for example by proteolytic cleavage with enzymes such as papain (to produce Fab fragments) or pepsin (to produce F(ab′)2 fragments). See e.g., Wahl et al., 24 J. Nucl. Med. 316-25 (1983). Portions of antibodies may be made by any of the above methods, or may be made by expressing a portion of the recombinant molecule. For example, the CDR region(s) of a recombinant antibody may be isolated and subcloned into the appropriate expression vector. See, e.g., U.S. Pat. No. 6,680,053.

The present invention concerns the novel monoclonal antibodies, designated the CA11-19 antibodies, which are specifically reactive with an antigen (EDP antigen) associated with human tumor cells, particularly from carcinomas of the lung, pancreas, and colon, methods for producing the CA11-19 monoclonal antibodies, and diagnostic and therapeutic methods employing the antibodies. The CA11-19 antibodies react with a range of tumors while showing essentially no reactivity with normal human tissues or other types of tumors such as melanomas or lymphomas.

The monoclonal antibodies of the present invention can be prepared by hybridoma fusion techniques. (see, Kohler and Milstein, Nature, 256:495-97 (1975); Brown et al., J. Immunol., 127 (2):539-46 (1981); Brown et al., J. Biol. Chem., 255:4980-83 (1980); Yeh et al., Proc. Nat'l. Acad. Sci. (USA), 76 (6):2927-31 (1976); and Yeh et al., Int. J. Cancer, 29:269-75 (1982)). These techniques involve the injection of an immunogen (e.g., purified antigen or cells or cellular extracts carrying the antigen) into an animal (e.g., a mouse) so as to elicit a desired immune response (i.e., production of antibodies) in that animal. In Example 1a preparation from human carcinoma of the colon designated is used as the immunogen. The tissue preparation is injected, for example, into a mouse, and after a sufficient time the mouse is sacrificed and somatic antibody-producing lymphocytes are obtained. Antibody-producing cells may be derived from the lymph nodes, spleens and peripheral blood of primed animals. Spleen cells are preferred. Mouse lymphocytes give a higher percentage of stable fusions with the mouse myelomas described below. The use of rat, rabbit and frog somatic cells is also possible. The spleen cell chromosomes encoding desired immunoglobulins are immortalized by fusing the spleen cells with myeloma cells, generally in the presence of a fusing agent such as polyethylene glycol (PEG). Any of a number of myeloma cell lines may be used as a fusion partner according to standard techniques; for example, the P3-NS1/1-Ag4-1, P3-x63-Ag8.653 or Sp2/O-Ag14 myeloma lines. These myeloma lines are available from the American Type Culture Collection (ATCC), Rockville, Md.

The resulting cells, which include the desired hybridomas, are then grown in a selective medium, such as HAT medium, in which unfused parental myeloma or lymphocyte cells eventually die. Only the hybridoma cells survive and can be grown under limiting dilution conditions to obtain isolated clones. The supernatants of the hybridomas are screened for the presence of antibody of the desired specificity, e.g., by immunoassay techniques using the antigen that has been used for immunization. Positive clones can then be subcloned under limiting dilution conditions, and the monoclonal antibody produced can be isolated. Various conventional methods exist for isolation and purification of the monoclonal antibodies so as to free them from other proteins and other contaminants. Commonly used methods for purifying monoclonal antibodies include ammonium sulfate precipitation, ion exchange chromatography, and affinity chromatography (see, e.g., Zola et al., in Monoclonal Hybridoma Antibodies: Techniques and Applications, Hurell (ed.) pp. 51-52 (CRC Press 1982)). Hybridomas produced according to these methods can be propagated in vitro or in vivo (in ascites fluid) using techniques known in the art (See, generally, Fink et al., supra, at page 123, FIG. 6-1).

Generally, the individual cell line may be propagated in vitro, for example in laboratory culture vessels, and the culture medium containing high concentrations of a single specific monoclonal antibody can be harvested by decantation, filtration or centrifugation. Alternatively, the yield of monoclonal antibody can be enhanced by injecting a sample of the hybridoma into a histocompatible animal of the type used to provide the somatic and myeloma cells for the original fusion.

It should be understood that the present invention encompasses the CA11-19 antibodies described herein and any fragments thereof containing the active binding region of the antibody, such as Fab, F(ab)₂ and Fv fragments. Such fragments can be produced from the CA11-19 antibodies using techniques well established in the art (see, e.g., Rousseaux et al., in Methods Enzymol., 121:663-69, Academic Press (1986)).

In addition, the present invention encompasses antibodies that are capable of binding to the same antigenic determinant as the CA11-19 antibodies and competing with the CA11-19 antibody for binding at that site. These include antibodies having the same antigenic specificity as the CA11-19 antibodies but differing in species origin, isotype, binding affinity or biological functions (e.g., cytotoxicity). For example, class, isotype and other variants of the antibody of the invention may be constructed using recombinant class-switching and fusion techniques known in the art (see, e.g., Thammana et al., Eur. L Immunol., 13:614 (1983); Spira et al., J. Immunol. Meth., 74:307-15 (1984); Neuberger et al., Nature, 312:604-08 (1984); and Oi et al., supra)). Thus, chimeric antibodies or other recombinant antibodies (e.g., antibody fused to a second protein such as a lymphokine) having the same binding specificity as the CA11-19 antibodies fall within the scope of this invention. Furthermore, since the EDP antigen to which the antibodies of the present invention binds is a novel tumor antigen, the antibodies of the invention includes antibodies that bind to any antigenic determinant on that EDP antigen, including determinants other than that with which the CA11-19 antibodies react.

Also included within the scope of the invention are anti-idiotypic antibodies of the CA11-19 antibodies. These anti-idiotypic antibodies can be produced using the CA11-19 antibodies as immunogen and are useful for diagnostic purposes in detecting humoral response to tumors and in therapeutic applications, e.g., in a vaccine, to induce an anti-tumor response in patients (See, e.g., Nepom et al., Cancer And Metastasis Reviews, 6:487-501 (1987); and Lee et al., Proc. Nat'l. Acad. Sci. (USA), 82:6286-90 (1985)).

For certain therapeutic applications chimeric (mouse-human) or human monoclonal antibodies may be preferable to murine antibodies because patients treated with mouse antibodies generate human antimouse antibodies. (Shawler et al., J. Immunol., 135:1530-35 (1985)). Human monoclonal antibodies may be made by using the EDP antigen of the invention, to sensitize human lymphocytes to the antigen in vitro followed by EBV-transformation or hybridization of the antigen-sensitized lymphocytes with mouse or human lymphocytes as described by Borrebaeck et al. (Proc. Nat'l. Acad. Sci. (USA), 85:3995-99 (1988)). Therefore, human monoclonal antibodies or chimeric antibodies that bind EDP antigen are also included within the scope of the present invention.

According to one embodiment, the antibodies of this invention, designated CA11-19, were produced via hybridoma techniques using tissue from colon carcinoma as the immunogen. The CA11-19 hybridomas, producing the CA11-19 antibodies, are in the possession of applicant at the time of filing this application and are being prepared for deposit with the American Type Culture collection (ATCC), 12301 Parklawn Drive, Rockville, Md. 20852. Accession numbers will be provided upon identification by ATCC.

The CA11-19 antibodies display very strong reactivity with tumor cells, particularly cells from colon, pancreas, and lung carcinomas. The antibodies of this invention do not display any immunohistologically detectable binding to normal human tissues such as fibroblasts, endothelial cells, or epithelial cells from the major organs.

The monoclonal antibodies of the invention are useful for diagnostic applications, both in vitro and in vivo, for the detection of human carcinomas carrying the EDP antigen with which the CA11-19 antibody is specifically reactive. In vitro diagnostic methods include immunohistological detection of tumor cells (e.g., on human tissue, cells or excised tumor specimens) and serologic detection of tumor-associated antigens (e.g., in blood samples or other biological fluids).

Immunohistological techniques involve contacting a biological specimen such as a tumor tissue specimen with the antibodies of the invention and then detecting the presence on the specimen of the antibodies complexed to their antigen. The formation of such antibody-antigen complexes with the specimen indicates the presence of tumor cells in the tissue. Detection of the antibodies on the specimen can be accomplished using techniques known in the art, such as the immunoperoxidase staining technique, the avidin-biotin (ABC) technique or immunofluorescence techniques (see, e.g., Ciocca et al., Meth. Enzymol., 121:562-79 (1986); Hellstrom et al., Cancer Research, 46:3917-23 (1986); and Kimball (ed.), Introduction To Immunology (2nd Ed.), pp. 113-117, Macmillan Publ. Co. (1986)).

Serologic diagnostic techniques involve the detection and quantitation of tumor-associated antigens that have been secreted or “shed” into the serum or other biological fluids of patients thought to be suffering from carcinoma. Such antigens can be detected in the body fluids using techniques known in the art such as radioimmunoassays (RIA) or enzyme-linked immunosorbent assays (ELISA) wherein an antibody reactive with the “shed” antigen is used to detect the presence of the antigen in a fluid sample (see, e.g., Uotila et al., J. Immunol. Methods, 42:11 (1981) and Allum et al. “Monoclonal Antibodies in the Diagnosis and Treatment of Malignant Conditions” Surg. Ann., 18:41-64, 48-51 (1986)). These assays, using the CA11-19 antibodies disclosed herein, therefore can be used for the detection in biological fluids of the EDP antigen with which the CA11-19 antibodies react and thus the detection of various carcinomas in human patients. Thus, it is apparent from the foregoing that the CA11-19 antibodies of the invention can be used in most assays involving antigen-antibody reactions. These assays include, but are not limited to, standard RIA techniques, both liquid and solid phase, as well as ELISA assays, immunofluorescence techniques, and other immunocytochemical assays (see, e.g., Sikora et al. (eds.), Monoclonal Antibodies, pp. 32-52, Blackwell Scientific Publications, (1984)).

The CA11-19 antibodies of the invention are also useful for in vivo diagnostic applications for the detection of human tumors. One such approach involves the detection of tumors in vivo by tumor imaging techniques using the antibodies labeled with an appropriate imaging reagent that produces detectable signal. Imaging reagents and procedures for labeling antibodies with such reagents are well known (see, e.g., Wensel and Meares, Radio Immunoimaging and Radioimmunotherapy, Esevier, N.Y. (1983); Colcher et al., Meth. Enzymol., 121:802-16 (1986)). The labeled antibodies may be detected by a technique such as radionuclear scanning (see, e.g., Bradwell et al. in Monoclonal Antibodies for Cancer Detection and Therapy, Baldwin et al. (eds.), pp. 65-85, Academic Press (1985)).

The CA11-19 antibodies of the invention have a number of in vivo therapeutic applications. In addition to being used alone to target tumor cells, the antibodies can be used in conjunction with an appropriate therapeutic agent to treat human cancer. For example, the antibodies can be conjugated or linked to a therapeutic drug or toxin for delivery of the therapeutic agent to the site of the cancer. Techniques for conjugating such therapeutic agents to antibodies are well known (see, e.g., Amon et al., Monoclonal Antibodies And Cancer Therapy, Reisfeld et al. (eds.), pp. 243-56, Alan R. Liss, Inc., (1985); Hellstrom et al in Controlled Drug Delivery (2nd ed.), Robinson et al. (eds.), pp. 623-53, Marcel Dekker, Inc., (1987); Thorpe, Monoclonal Antibodies '84: Biological And Clinical Applications, Pinchera et al. (eds.), pp. 475-506 (1985); and Thorpe et al., Immunol. Rev., 62:119-58 (1982)).

Alternatively, the antibodies can be coupled to a source of high-energy radiation, e.g., a radioisotope such as ¹³¹I, which, when localized at the tumor site, results in a killing of several cell diameters (See, e.g., Order, in Monoclonal Antibodies For Cancer Detection And Therapy, Baldwin et al. (eds.), pp. 303-16, Academic Press, (1985)).

Furthermore, chimeric or other recombinant CA11-19 antibodies of the invention, as described earlier, may be used therapeutically. For example, a fusion protein comprising at least the antigen-binding region of the CA11-19 antibodies joined to at least a functionally active portion of a second protein having anti-tumor activity, e.g., a lymphokine or oncostatin, may be used to treat human tumors in vivo. In addition, a chimeric CA11-19 antibody wherein the antigen-binding region of CA11-19 is joined to a human Fc region, e.g., IgG1, may be used to promote antibody-dependent cellular cytotoxicity or complement mediated cytotoxicity.

It is apparent, therefore, that the present invention encompasses pharmaceutical compositions, combinations and methods for treating human tumors. For example, the invention includes pharmaceutical compositions for use in the treatment of human tumors comprising a pharmaceutically effective amount of a CA11-19 antibodies and a pharmaceutically acceptable carrier. The compositions may contain the CA11-19 antibodies, either unmodified, or conjugated to a therapeutic agent (e.g., drug, toxin, enzyme or second antibody). The compositions may additionally include other antibodies or conjugates for treating carcinomas (e.g., an antibody cocktail).

The antibody compositions of the invention can be administered using conventional modes of administration, including, but not limited to, intravenous, intraperitoneal, oral, intralymphatic or administration directly into the tumor. Intravenous administration is preferred.

The antibody compositions of the invention may be in a variety of dosage forms which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The antibody compositions also preferably include conventional pharmaceutically acceptable carriers and adjuvants known in the art such as human serum albumin, ion exchangers, alumina, lecithin, buffer substances such as phosphates, glycine, sorbic acid, potassium sorbate, and salts or electrolytes such as protamine sulfate.

The present invention also concerns the novel antigen, referred to as EDP antigen. EDP antigen can be used purified via the methods set forth below. EDP antigen may be used for therapeutic applications. For example, the purified EDP antigen may be administered alone as an immunogen or together with a proper immunological adjuvant.

In order that the invention described herein may be more fully understood, the following examples are set forth. It should be understood that these examples are for illustrative purposes only and are not to be construed as limiting the scope of this invention in any manner.

### Example I

### Isolation and Characterization of EDP Antigen

Tumor carcinomas of the gastrointestinal tract were minced, homogenized and extracted with 1.4M perchloric acid for 60 minutes and then centrifuged at 10,000 rpm for 30 minutes. The extract supernatant was extensively dialyzed against water, lyophlized and reconstituted to suitable concentrations with PBS pH 7.5. Protein band MW ˜60,000 daltons was isolated by preparative PAGE electrophoresis (7.5% acrylamide gels) and further absorbed of contaminating glycoproteins by CNBr-Sepharose 48 affinity chromatography using anti-whole normal serum; ‘anti-aI-acid glycoprotein, anti-01-antitrypsin, anti-albumin and anti-CEA, proteins which were found to co-extract with perchloric acid treatment.

Antigen purified in this manner was then injected into rabbits. After a suitable antibody titer was developed in the rabbits further isolation began with the perchloric acid extraction described above. Supernatant was then purified via affinity chromatography using the anti-EDP antigen antibodies obtained from the rabbits. At this point the affinity columns against other proteins, described above, were used again. Antigen isolated by both methods demonstrate single band purity by SDS-PAGE analysis.

EDP antigen has a molecular weight of about 60,000 Daltons with a secondary band at 36,000 Daltons when subjected to polyacrylamide gel electrophoresis. Thin layer isoelectric focusing reveals that EDP antigen has a pI of 4.5 in polyamylamide gels with a pH gradient from 3 to 8. EDP antigen has a peak UV absorbance at 230 nm wavelength.

EDP antigen was labeled with iodine-125 using the Iodogen method (Pierce Biochemicals). Radiolabeled EDP antigen showed a single band on sliced gel eluates obtained from SDS-PAGE electrophoresis of the labeled antigen.

The N-terminus sequence of EDP antigen is: SEQ ID NO: 1. While sequencing of the N-terminus of EDP antigen was clear for several residues, some residues in the sequence yielded results leaving uncertainty as to one out of two possible amino acids for a given residue. SEQ ID NO: 2 represents the N-terminus amino acid sequence of EDP antigen with all minor yield amino acids replacing the major yield amino acids. Some combination of SEQ ID NO: 1 and SEQ ID NO: 2 represents the The N-terminus amino acid sequence of EDP antigen.

### Example II

### Preparation of the CA11-19 Monoclonal Antibody

The CA11-19 monoclonal antibody of the invention was produced using hybridoma fusion techniques described previously.

### Example III

### Immunohistochemical Staining of Tissue Samples

Tissue sections derived from Adenocarcinoma of the colon, normal colon tissue adjacent to the neoplastic site, fetal liver and fetal lung sections were all examined using direct immunofluorescent techniques for the presence of EDP antigen and for the presence of CEA.

The antibodies (Monoclonal and Polyclonal) to Tennessee Antigen and CEA (DAKO, Denmark) were separately labeled with fluorescein isothiocyanate.

Examination of the sections by means of fluorescence microscopy demonstrated that the primary staining of the malignant tissue was associated mainly with cell plasma membranes. No staining above background was evident in sections of adult lung tissue. The staining seen in the malignant tissue using fluorescently-tagged anti CEA serum was similar to the pattern seen with anti EDP antigen stained with CA11-19 antibodies.

Cross-inhibition studies demonstrated that unlabeled anti-CEA serum did not inhibit staining for EDP antigen by fluorescently-tagged anti EDP antigen serum. Also, unlabeled anti-EDP antigen serum did not inhibit the staining by the labeled anti-CEA serum.

### Example IV

### Serum Assay for EDP Antigen

The distribution of EDP antigen values in serum of patients with various malignant and non-malignant diseases, as well as in healthy subjects, was determined in over 5,788 individuals from eighteen institutions. Subjects included those suspected of having cancer as well as those with other medical problems. The study included a group of healthy blood donors, some of whom were smokers. Smokers were defined as smoking at least one pack of cigarettes per day. All diagnoses listed as malignancy were histologically confirmed. Subjects indicating positive for cancer included represented a spectrum of disease states.

5 ml samples of human blood was collected from all subjects using standard venipuncture techniques. The samples were allowed to clot and the serum was separated from the clot by centrifugation. Complete clot formation was allowed prior to centrifugation to prevent erroneous results due to the presence of fibrinogen.

96-well plates were coated with CA11-19 antibodies and allowed to dry. 25 μl of Patient Serum Specimens and 75 μl of Sample Diluting Buffer were pipette into their assigned wells. Plates were sealed and incubated at 37° C.+/−2° C. incubator overnight, a minimum of 12 hours to a maximum of 24 hours. Wells were aspirated and washed three times with deionized water.

100 μl of Goat Anti-CA11-19 Alkaline Phosphastase Conjugate was added to each well. The plates were sealed and incubated for 2 hours at 37° C.+/−2° C. and washed three times with deionized water. 100 μl Substrate Solution was added to each well and the plates were incubated at 37° C.+/−2° C. for a minimum of 30 minutes. Following the latest incubation optical density at 410 or 405 nm was determined on an ELISA plate reader. Plates were periodically read at ten minute intervals looking for optical density reading of CA11-19 STANDARD I (20 u/ml) to be 1.00 or greater. Incubation was continued until CA1-18 STANDARD I (20 u/ml) optical density reached 1.0 or greater. 50 μl of 3N NaOH was dispensed in each well when CA1-18 STANDARD I (20 u/ml) reading reached 1.00 optical density at 410 or 405 nm wavelength. All wells were read at 410 or 405 nm wavelength.

Final concentrations for Sample Diluting Buffer were:

A. Enzyme Phosphate Buffered Saline

| | | |
|---|---|---|
| 1. | Sodium Chloride | 0.8% |
| 2. | Potassium Phos. (Mono) | 0.02% |
| 3. | Sodium Phos. (Dibasic) | 0.115% |
| 4. | Potassium Cl | 0.02% |
| 5. | Sodium Azide | 0.02% |
| | in sterile Distilled H₂0 | |

| | |
|---|---|
| B. Tweene 20 | 0.05% |
| C. Bovine Serum Albumin (Fraction V) | 0.5% |
| D. Normal Rat Serum | 2-4% |

(each batch (lot number) has to be titered against panel of known serums)

Final concentrations for Sample Substrate Solution were:

| | |
|---|---|
| A. Diethanolamine | 10% |
| B. Magnesium Chloride | 0.1% |
| C. Sodium Chloride | 0.2% |

D. HCL Concentrate, to stabilize pH at 9.8

Results using the CA1-18 EIA Assay measuring Tennessee Antigen directly in serum samples from cancer patients, active non-cancer patients and normal individuals (no evidence of disease)

TABLE 1

| Subject category | No. | % Neg | % Pos |
|---|---|---|---|
| Cancer Patients* | 33 | 18.0% | 82.0% |
| Non-Cancer Diseases | 19 | 79.0% | 21.0% |
| Normal Individuals | 26 | 92.3% | 7.7% |

*Cancer patients included those with Carcinoma of Colon, Rectum, Lung, Breast and Pancreas.

It is apparent that many modifications and variations of this invention as set forth above may be made without departing from the spirit and scope. The specific embodiments described are given by way of example only, and the invention is limited only by the terms of the appended claims.

What is claimed is:

1. A monoclonal antibody produced by a hybridoma cell line, which antibody binds to a determinant site on an antigen of human tumor cells, said antigen characterized by a molecular weight of about 60,000 Daltons, as determined by polyacrylamide gel electrophoresis, and having a terminal amino acid sequence comprising SEQ ID NO: 1.

2. The monoclonal antibody of claim 1 wherein said tumor cells are carcinoma cells.

3. The monoclonal antibody of claim 2 wherein said carcinoma cells are selected from the group consisting of lung, pancreas, and colon carcinoma cells.

4. The monoclonal antibody of claim 1 conjugated to a label capable of producing a detectable signal.

5. The monoclonal antibody of claim 4 wherein said label is selected from the group consisting of a radionuclide, an enzyme, a fluorescent agent and a chromophore.

6. The monoclonal antibody of claim 1 which is a murine antibody.

7. The monoclonal antibody of claim 1 which is a human antibody.

8. The monoclonal antibody of claim 1 which is a mouse-human antibody.

9. An immunoassay for the detection of EDP antigen comprising:
a) combining one or more monoclonal antibodies reactive with an antigen associated with human tumor cells, said antigen characterized by a molecular weight of about 60,000 daltons as determined by polyacrylamide gel electrophoresis and having an amino terminal amino acid sequence comprising SEQ ID NO: 1, with a human tissue or fluid sample; and
b) assaying for binding of EDP antigen to said one or more monoclonal antibodies.

10. The immunoassay of claim 9 wherein said monoclonal antibodies are the CA11-19 antibodies produced by hybridoma cell lines deposited with ATCC.

11. The immunoassay of claim 9 wherein said monoclonal antibodies are labeled with a label selected from the group consisting of a radionuclide, an enzyme, a fluorescent agent and a chromophore.

12. The immunoassay of claim 9 wherein said binding of EDP antigen to said one or more monoclonal antibodies is detected by ELISA assay.

13. The method of claim 9 wherein said binding of EDP antigen to said one or more monoclonal antibodies is detected by adding additional antibodies capable of binding EDP antigen.

14. The method of claim 13 wherein said additional antibodies capable of binding EDP antigen are labeled with a label selected from the group consisting of a radionuclide, an enzyme, a fluorescent agent and a chromophore.

15. The method of claim 9 wherein said assaying for binding of EDP antigen to said one or more monoclonal antibodies is performed by immunohistological staining.

16. A continuous cell line which produces a monoclonal antibody which specifically binds a determinant site on a cell surface glycoprotein antigen associated with tumor cells, said antigen having a molecular weight of about 60,000 Daltons and having a terminal amino acid sequence comprising SEQ ID NO: 1.

17. The continuous cell line deposited with ATCC.

18. An isolated protein comprising EDP antigen, wherein said protein includes the amino acid sequence of SEQ ID NO: 1 in the N-terminal portion of said protein, and can be found co-localized with plasma membranes of human colorectal carcinoma, and has a peak UV absorbance at approximately 230 nm wavelength, and said protein shows a molecular weight of about 60 kDa with a secondary band of molecular weight 36 kDa when measured by SDS-PAGE.

19. The isolated protein of claim 18 wherein said protein is present on the surface of human carcinomas.

20. The isolated protein of claim 18 wherein said human carcinomas is colon carcinoma.
